(19) United States
(12) Patent Application Publication
(10) Pub. No.: US 2014/0154335 A1
(43) Pub. Date: Jun. 5, 2014

(54) ANTIMICROBIAL PACKAING

(71) Applicant: Whitehall Products, LLC, Tampa, FL (US)

(72) Inventor: Michael Russo, Pinellas Park, FL (US)

(73) Assignee: Whitehall Products, LLC, Tampa, FL (US)

(21) Appl. No.: 14/093,745

(22) Filed: Dec. 2, 2013

Related U.S. Application Data
(60) Provisional application No. 61/733,032, filed on Dec. 4, 2012.

(51) Int. Cl. A61K 47/32; A61K 45/06; A61K 33/38

(52) U.S. Cl. CPC A61K 47/32; A61K 33/38; A61K 45/06
USPC 424/619

(57) ABSTRACT

An antimicrobial packaging polymer and its method of use, and more particularly to a contact antimicrobial such as metal or metal ions covalently bound to a polymeric material that may be suitable in a variety of applications such as film and container packaging of perishable items, medical equipment and devices, environmental, hygienic and sanitary applications, as well as other consumer and commercial use.

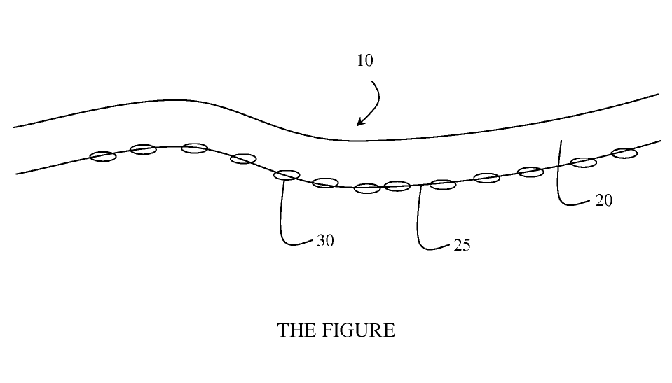

### CROSS REFERENCE TO RELATED APPLICATIONS

This application is a Non-Provisional application of co-pending U.S. Provisional Application No. 61/733,032 filed Dec. 4, 2012, which is incorporated herein by reference.

### FIELD OF INVENTION

This invention relates to a polymeric antimicrobial packaging material.

### BACKGROUND OF THE INVENTION

The commercial application of antimicrobial packaging has been an area of interest for many years. The motivation behind antimicrobial packaging is to enhance, not replace, the safety and quality measures already used by the food industry. It is also a goal to extend the sanitary conditions found in the commercial environment to the consumer environment.

Several factors should be considered when designing an antimicrobial packaging system. Regulatory approval of the antimicrobial agent is, of course, a critical factor. Some antimicrobial systems rely on diffusion or release of the antimicrobial agent in order for it to be effective. This would place the packaging material under the classification of an indirect food additive and the material would require review by the US Food and Drug Administration (FDA) along with similar scrutiny in other countries. Generally, approval may be obtained if the additive has already been effectively used as a direct food additive as long as migratory concentrations and conditions of use are addressed.

Another critical factor is production cost. Some antimicrobial systems may be effective, but production costs may negate the benefits obtained by an extended shelf life or improvement in quality. Lastly, there are numerous technical challenges related to coating methods, the rate of curing, the ease of heat sealing, the effects on physical and mechanical properties of film, the effects on color, the texture or flavor of the food as well as the ability of the antimicrobial agent to provide effectiveness throughout the package/product life cycle. (See K. Cooksy, Effectiveness of antimicrobial food packaging materials, Food Additives and Contaminants, October, 2005; 22(10): 980-987).

A variety of antimicrobial packaging systems have been reviewed (Kesler and Fennema 1986; Krochta and De Mulder-Johnston 1997; Han 2000; Cooksey 2001; Brody 2002). While some films have incorporated the antimicrobial agent into the polymer (Siragusa et al. 1999), others have used biopolymer films as effective carriers of antimicrobial agents (Padgett et al. 1998; Coma et al. 2001). Many of these biopolymer films are cellulose-based, and because of their water-soluble nature, they effectively release additives when combined with foods of high water content. For example, upon contact, a cellulose-based matrix degrades and releases the antimicrobial agent from the matrix to the surface of the food product resulting in bacterial inhibition. Silver nitrate (AgNO₃), however, is an inorganic compound with inherent antimicrobial properties. Silver nitrate is also colorless and relatively non-reactive to light.

### SUMMARY OF INVENTION

The present invention is an anti-microbial polymeric film that has a substrate that includes at least one surface that incorporates an antimicrobial compound, which antimicrobial compound is between 1% and 10% by weight of the antimicrobial polymeric film.

Another embodiment of the present invention is an anti-microbial polymeric film that has a substrate that includes at least one surface that incorporates an antimicrobial compound, which antimicrobial compound is between 1% and 10% by weight of the antimicrobial polymeric film and where the antimicrobial compound is in particulate form.

Another embodiment of the present invention is anti-microbial polymeric film that has a substrate that includes at least one surface that incorporates an antimicrobial compound, which antimicrobial compound is between 1% and 5% by weight of the antimicrobial polymeric film.

Another embodiment of the present invention is an anti-microbial polymeric film that has a substrate that includes at least one surface that incorporates an antimicrobial compound, which antimicrobial compound is between 2% and 4% by weight of the antimicrobial polymeric film.

Another embodiment of the present invention is an anti-microbial polymeric film that has a substrate that includes at least one surface that incorporates an antimicrobial compound, which antimicrobial compound is an inorganic compound comprising a metal or metal ions selected from the group consisting of silver, copper, zinc, tin, mercury, lead, cobalt, nickel, manganese, arsenic, antimony, bismuth, barium, cadmium, chromium and combinations thereof.

Another embodiment of the present invention is an anti-microbial polymeric film that has a substrate that includes at least one surface that incorporates an antimicrobial compound, which antimicrobial compound is an inorganic compound comprising a metal or metal ions, and such inorganic compound is between about 1% and 50%, by weight of the antimicrobial compound.

Another embodiment of the present invention is an anti-microbial polymeric film that has a substrate that includes at least one surface that incorporates an antimicrobial compound, which antimicrobial compound is an inorganic compound comprising a metal or metal ions, and such inorganic compound is between about 1% and 30%, by weight of the antimicrobial compound.

Another embodiment of the present invention is an anti-microbial polymeric film that has a substrate that includes at least one surface that incorporates an antimicrobial compound, which antimicrobial compound is an inorganic compound comprising a metal or metal ions, and such inorganic compound is between about 1% and 15%, by weight of the antimicrobial compound.

Another embodiment of the present invention is an anti-microbial polymeric film that has a substrate that includes at least one surface that incorporates an antimicrobial compound, which antimicrobial compound is an inorganic compound comprising a metal or metal ions, and such inorganic compound is between about 15% and 20%, by weight of the antimicrobial compound.

Another embodiment of the present invention is an anti-microbial polymeric film that has a substrate that includes at least one surface that incorporates an antimicrobial compound, wherein the substrate is a polyester, polyethylene, polyolefin, polyamide and PVC.

### BRIEF DESCRIPTION OF THE DRAWINGS

The FIGURE is a diagrammatic representation of an illustrative embodiment of the invention.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The antimicrobial polyolefin resin composition of the present invention may be a resin material for obtaining antimicrobial polyolefin resin molded articles, or a master batch for obtaining the resin material. Namely, when the composition is pellet or particle obtained by kneading and, for example, pelletizing or pulverizing a mixture which is obtained by mixing components such as the antimicrobial agent, the vehicle and optionally additives described after, with a small amount of the thermoplastic polyolefin resin as a base resin, the composition of the present invention is usually a master batch. On the other hand, when the composition is a composition obtained by kneading and diluting the above-described master batch with a thermoplastic polyolefin resin as a base resin, the composition of the present invention is a resin material.

The inorganic antimicrobial agent used in the present invention is not particularly limited, and, for example, (i) an antimicrobial agent prepared by supporting at least one metal ion selected from the group consisting of a silver ion, cupper ion, zinc ion and tin ion, on zeolite, (ii) an antimicrobial agent containing a phosphorous salt containing a silver ion, as an effective ingredient, (iii) an antimicrobial agent composed of fusible powder containing a silver ion, and the like are illustrated. Among these antimicrobial agents, an antimicrobial agent which is prepared by supporting a silver ion is preferred. The number average particle diameter of the inorganic antimicrobial agent is not particularly limited.

The content of the inorganic antibacterial agent in the antibacterial polyolefin resin composition is not particularly limited. For example, in one embodiment, the inorganic antibacterial agent is between about 1% and 50%, by weight of the antimicrobial compound. In another embodiment, the inorganic antibacterial agent is between about 1% and 30%, by weight of the antimicrobial compound. Another embodiment contemplates a film wherein the inorganic agent is between about 1% and 15%, by weight of the antimicrobial compound. Lastly, the inorganic agent can be between about 15% and 20%, by weight of the antimicrobial compound. Without limiting the invention, the term “about” as used herein includes variations from the given examples that do not materially affect the basic results discussed below or the novelty of the claimed invention.

In a preferred embodiment, however, when the composition of the present invention is a master batch, the content of the inorganic antibacterial agent based on the composition (master batch) is preferably about 1 to 10% by weight, more preferably about 3 to 5% by weight. Namely, a preferable mode of the antibacterial polyolefin resin composition is a master batch containing 3% by weight of the antibacterial agent based on the master batch. In the present invention, the composition can disperse and contain the antibacterial agent of high concentration such the range mentioned above because the dispersability of the antibacterial agent is advantageous.

In addition, when the composition is a resin material, in other words, when the composition is used for molding to produce molded articles without diluting the composition with a base polyolefin resin, the concentration of the antimicrobial agent may be properly determined taking account of use of the composition and the like.

In the present invention, the thermoplastic polyolefin resin as the base resin is not particularly restricted, and there are illustrated thermoplastic polyolefin resins which are exemplified as the vehicle (with proviso that the number average molecular weight is not restricted because the resins are used in different use from the vehicle) and have a softening point higher than that of the low molecular weight thermoplastic polyolefin resin as the vehicle. In addition, the thermoplastic polyolefin resin as the base resin may be crystalline or non-crystalline. The kind of the base resin used may be the same as or different from that of the vehicle, but is preferably the same kind as the vehicle. The base resin may be one kind or a mixture of two or more kinds of thermoplastic polyolefins.

The antimicrobial thermoplastic polyolefin resin of the present invention optionally also contain various additives within a range in which the effect of the present invention is not damaged. The additives are used for the purpose of improving resin properties such as processability, flexibility, elasticity, brittleness, manageability, etc., resin performance such as stability, durability, inflammability, heat insulation ability, etc., workability such as mold releasibility, kneading ability, etc., and are not particularly limited unless they are decomposed in the molten resin. Examples thereof include a plasticizer, antioxidant, UV absorber, light stabilizer, flame retardant, antistatic agent, copper inhibitor, metal deactivator, lubricant, slip agent, internal mold releasing agent, defogging agent, perfuming agent, surfactant, wetting agent, preservative, mildew resistance agent, filler, reinforcing agent, stabilizer, heat insulator, foaming agent, anti-dumping material, impact resistance improver, surface treating agent, dispersing agent, etc. The additive used may be one kind or more.

The antimicrobial thermoplastic polyolefin resin composition of the present invention as a resin material can be obtained by heating and melting the master batch together with a thermoplastic polyolefin resin to dilute the master batch. The antimicrobial thermoplastic polyolefin resin composition of the present invention is useful for use of, for example, fibers, films, molded articles and the like.

Therefore, according to an illustrative embodiment of the invention, as shown in The FIGURE, the inventive includes antimicrobial polymeric film 10 comprising a polymeric substrate 20 having at least one surface 25 having an antimicrobial compound. In the illustrative embodiment shown in The FIGURE, antimicrobial compound 30 is in particulate form.

### EXAMPLES

The present invention will be described in more detail referring to Examples below, but is not limited thereto. The following examples are based on an illustrative embodiment of the invention comprising a 2M antimicrobial storage bag having the following properties:

| | | | | Weight |
|---|---|---|---|---|
| Width | Length | Gauge | Count | (lbs.)/(kg) |
| 13″ | 15″ | 0.0015 | 2,000,000 | 39,000/17,690 |

The thermoplastic polyolefin composition of this illustrative embodiment was as follows:

| Item | % (Weight) | Weight (lbs.)/(kg) | |
|---|---|---|---|
| Linear Low | 97% | 25,220 | (lbs.) |
| Density | | 11,439 | (kg) |
| Polyethylene | | | |
| Compounded | 3% | 780 | (lbs.) |
| Silver Nitrate | | 345 | (kg) |
| (AgNO₃) | | | |
| Silver Nitrate | 15% of | 117 | (lbs.) |
| (AgNO₃) | Compound | 56 | (kg) |

### Example I

To test the efficacy of the illustrative embodiment of the invention, testing was conducted according to the methodologies outlined in the FDA Bacteriological Analytical Manual, and Compendium of Methods for the Microbial Examination of Food, 4^{th} Edition. Tests were performed on one (1) private label turkey breast and one (1) national brand turkey breast, each split into three portions. One portion of each sample was wrapped with plastic not comprising an antimicrobial agent and one portion was placed in a bag comprising the illustrative embodiment.

Conditions were established to approximate the conditions of commercial establishment, such as a delicatessen. Each portion was removed, twice per day, from the end exposed to ambient air conditions for four (4) minutes while being sliced from one end only. After slicing, each portion was returned to its container (plastic wrap or illustrative embodiment).

The following table reveals the testing results for the national turkey brand:

| National | | | | | | | |
|---|---|---|---|---|---|---|---|
| Turkey Brand | TIME PERIOD OF ANALYSIS | | | | | | |
| ASSAY | Day 0 | Day 4 | Day 6 | Day 7 | Day 8 | Day 9 | Day 10 |
| APC/g | 57,000 | <10 | <10 | <10 | <10 | <10 | <10 |
| Lactic Acid | <10 | <10 | <10 | <10 | <10 | <10 | <10 |
| Bacteria/g | | | | | | | |
| Yeast/g | <10 | <10 | <10 | <10 | <10 | <10 | <10 |
| Psychrotroph/g | <10 | <10 | <10 | <10 | <10 | <10 | <10 |
| Visual | Normal | Normal | Normal | Normal | Normal | Normal | Normal |
| Description | | | | | | | |
| Odor | Normal | Normal | Normal | Normal | Normal | Normal | Normal |
| Description | | | | | | | |

The following table reveals the testing results for the private label turkey brand:

| Private Label | | | | | | | |
|---|---|---|---|---|---|---|---|
| Turkey | TIME PERIOD OF ANALYSIS | | | | | | |
| ASSAY | Day 0 | Day 4 | Day 6 | Day 7 | Day 8 | Day 9 | Day 10 |
| APC/g | 7,100 | <10 | 720 | <10 | <10 | <10 | <10 |
| Lactic Acid | <10 | <10 | 560 | <10 | <10 | <10 | <10 |
| Bacteria/g | | | | | | | |
| Yeast/g | <10 | <10 | <10 | <10 | <10 | <10 | <10 |
| Psychrotroph/g | <10 | <10 | <10 | <10 | <10 | <10 | <10 |
| Visual | Normal | Normal | Normal | Normal | Normal | Normal | Normal |
| Description | | | | | | | |
| Odor | Normal | Normal | Normal | Normal | Normal | Normal | Normal |
| Description | | | | | | | |

### Example II

To test the efficacy of the illustrative embodiment of the invention, testing was conducted according to the methodologies outlined in the FDA Bacteriological Analytical Manual, and Compendium of Methods for the Microbial Examination of Food, 4^{th} Edition. Tests were performed on one (1) private label block of cheese and one (1) national brand block of cheese. One portion of each sample was hand-wrapped with plastic not comprising an antimicrobial agent and one portion was placed in a bag comprising the illustrative embodiment.

Conditions were established to approximate the conditions of commercial establishment, such as a delicatessen. Each portion was removed, twice per day, from the end exposed to ambient air conditions for four (4) minutes while being sliced from one end only. After slicing, each portion was returned to its container (plastic wrap or illustrative embodiment).

After analysis, yeast and mold counts were not detected through all time points up to twenty two (22) days of the shelf-life study. The following table reveals the testing results for the national cheese sample:

| National | | | | | | | |
|---|---|---|---|---|---|---|---|
| Brand Cheese | TIME PERIOD OF ANALYSIS | | | | | | |
| ASSAY | Day 0 | Day 7 | Day 14 | Day 16 | Day 18 | Day 20 | Day 22 |
| Yeast/g | <10 | <10 | <10 | <10 | <10 | <10 | <10 |
| Mold/g | <10 | <10 | <10 | <10 | <10 | <10 | <10 |
| Visual | Normal | Normal | Normal | Normal | Normal | Normal | Normal |
| Description | | | | | | | |
| Odor | Normal | Normal | Normal | Normal | Normal | Normal | Normal |
| Description | | | | | | | |

The following table reveals the testing results for the private label cheese brand:

| Private Label | | | | | | | |
|---|---|---|---|---|---|---|---|
| Cheese | TIME PERIOD OF ANALYSIS | | | | | | |
| ASSAY | Day 0 | Day 4 | Day 6 | Day 7 | Day 8 | Day 9 | Day 10 |
| Yeast/g | <10 | <10 | <10 | <10 | <10 | <10 | <10 |
| Mold/g | <10 | <10 | <10 | <10 | <10 | <10 | <10 |
| Visual | Normal | Normal | Normal | Normal | Normal | Normal | Normal |
| Description | | | | | | | |
| Odor | Normal | Normal | Normal | Normal | Normal | Normal | Normal |
| Description | | | | | | | |

### Example III

Another analysis was performed under the conditions of Example II, replacing the 3% antimicrobial additive with a 2% antimicrobial additive. E. coli colony formation results were as follows:

| | Inoculation | | | | |
|---|---|---|---|---|---|
| Organism | Level | Day 0 | Day 7 | Day 14 | Day 28 |
| E. coli ATCC | 4.1 × 10⁶ | 6 × 10⁴ | 50 | <10 | 330 |
| 0157 | | | | | |
| E. coli ATCC | 2.1 × 10⁷ | 2.1 × 10⁵ | <10 | <10 | <10 |
| 0111 | | | | | |
| S. Aureus | 3.9 × 10⁶ | 3 × 10⁵ | <10 | <10 | <10 |
| E. coli | 4.6 × 10⁶ | 4.2 × 10⁵ | 7.9 × 10² | <10 | <10 |
| Ps. Aeruginosa. | 5.4 × 10⁶ | 4.4 × 10⁵ | 4 × 10⁶ | 2.57 × 10⁶ | 1 × 10⁵ |
| ATCC 9027 | | | | | |
| C. albicans | 2.08 × 10⁶ | 4.1 × 10⁴ | 2 × 10² | <10 | <10 |
| ATCC 10231 | | | | | |
| A. niger | 1.1 × 10⁶ | 2 × 10³ | <10 | <10 | <10 |

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. An antimicrobial polymeric film comprising a polymeric substrate having at least one surface having an antimicrobial compound, wherein said antimicrobial compound is between about 1% and 10%, by weight of the antimicrobial polymeric film.

2. The antimicrobial polymeric film of claim 1, wherein said antimicrobial compound is in particulate form.

3. The antimicrobial polymeric film of claim 1, wherein said antimicrobial compound is between about 1% and 5%, by weight of the antimicrobial polymeric film.

4. The antimicrobial polymeric film of claim 1, wherein said antimicrobial compound is between about 2% and 4%, by weight of the antimicrobial polymeric film.

5. The antimicrobial polymeric film of claim 1, wherein said antimicrobial compound further comprises an inorganic compound comprising a metal or metal ions selected from the group consisting of silver, copper, zinc, tin, mercury, lead, cobalt, nickel, manganese, arsenic, antimony, bismuth, barium, cadmium, chromium and combinations thereof.

6. The antimicrobial polymeric film of claim 5, wherein the inorganic compound comprises AgNO₃.

7. The antimicrobial polymeric film of claim 5, wherein said inorganic compound is between about 1% and 50%, by weight of the antimicrobial compound.

8. The antimicrobial polymeric film of claim 5, wherein said inorganic compound is between about 1% and 30%, by weight of the antimicrobial compound.

9. The antimicrobial polymeric film of claim 5, wherein said inorganic compound is between about 1% and 15%, by weight of the antimicrobial compound.

10. The antimicrobial polymeric film of claim 5, wherein said inorganic compound is about 15% and 20%, by weight of the antimicrobial compound.

11. The antimicrobial polymeric film of claim 1, wherein said polymeric substrate is selected from the group consisting of polyester, polyolefin, polyamide and PVC.

12. An antimicrobial polymeric film comprising:
a. a polymeric substrate having at least a first surface and an opposing second surface; and
b. an antimicrobial compound disposed on the first surface of the polymeric substrate;
c. wherein the polymeric substrate is comprised of a linear low density polyethylene; and
d. wherein the antimicrobial compound comprises an inorganic compound comprising a metal or metal ions.

13. The antimicrobial polymeric film of claim 12, wherein the linear low density polyethylene is about 97%, by weight, of the polymeric film.

14. The antimicrobial polymeric film of claim 12, wherein the antimicrobial compound is about 3%, by weight, of the polymeric film.

15. The antimicrobial polymeric film of claim 14, wherein the metal or metal ions comprise about 15%, by weight, of the antimicrobial compound.

16. The antimicrobial polymeric film of claim 12, wherein said antimicrobial compound further comprises an inorganic compound comprising a metal or metal ions selected from the group consisting of silver, copper, zinc, tin, mercury, lead, cobalt, nickel, manganese, arsenic, antimony, bismuth, barium, cadmium, chromium and combinations thereof.

17. The antimicrobial polymeric film of claim 12, wherein the inorganic compound comprises AgNO₃.

18. The antimicrobial polymeric film of claim 12, wherein said antimicrobial compound is in particulate form.
